(19) United States
(12) Patent Application Publication
(10) Pub. No.: US 2009/0264805 A1
(43) Pub. Date: Oct. 22, 2009

(54) Incision bandage

(76) Inventors: Joyce Davis, Lexington, KY (US); Dawn Bell, Charleston, WV (US)

Correspondence Address: STOCKWELL & SMEDLEY, PSC, 861 CORPORATE DRIVE, SUITE 200, LEXINGTON, KY, 40503, US

(21) Appl. No.: 12/148,146

(22) Filed: Apr. 17, 2008

(51) Int. Cl. A61F 13/15; A61F 13/02

(52) U.S. Cl. 602/43; 602/54

(57) ABSTRACT

Embodiments of the present invention relate to a bandage that has a transparent portion that also includes an arcuate scale for viewing a portion of a wound site underneath the bandage. Additionally, the backing of the bandage may be transparent as well, with a similar arcuate scale, that a user may use after the bandage is removed.

### BACKGROUND OF THE INVENTION

1. Field of the Invention

The present invention relates generally to a bandage for an incision site and, more particularly, to a bandage having a curved measuring scale imprinted thereon.

2. Description of Related Art

There are a variety of bandages and dressing that have been developed for placing over wounds to protect them during the healing process. Most of these bandages have some sort of adhesive backing that allows them to stick to the wound site with varying degrees of strength depending on their intended use. Some of these dressings are opaque in nature such that the gauze or padding along with the bandage completely visually block the area of the body which they cover. In other instances a portion of the bandage may be constructed from a transparent material so that at least part of the wound site is visible even with the bandage present.

In some instances, transparent bandages have been developed that have rectangular grids that can be used to monitor a wound site. In operation, a trained professional observes the bandage and wound site and estimates the area of some type of trauma that may be visible through the bandage. More particularly, each grid square that is occupied by the trauma is counted and then each grid square that is partially occupied is estimated in some subjective way to arrive at a total measure of the trauma. This procedure requires trained personnel, is very time consuming, and has proven to provide highly varying results based on different people doing the estimating.

Another similar device used in treating chronic sores and wounds is a transparent score card which a practitioner places temporarily over the wound site and then traces on the score card the outline of the wound. This score card is then removed and either added to a patient's files or analyzed further so that appropriate annotations can be added to a patient's file.

There exists the need therefore for a bandage that can be used by untrained personnel to accurately and repeatedly measure an incision site quickly and easily.

### BRIEF SUMMARY OF THE INVENTION

Embodiments of the present invention relate to a bandage that has a transparent portion that also includes an arcuate scale for viewing a portion of a wound site underneath the bandage. Additionally, the backing of the bandage may be transparent as well, with a similar arcuate scale, that a user may use after the bandage is removed.

It is understood that other embodiments of the present invention will become readily apparent to those skilled in the art from the following detailed description, wherein it is shown and described only various embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF DRAWINGS

Various aspects of a transparent bandage are illustrated by way of example, and not by way of limitation, in the accompanying drawings, wherein:

FIG. 1 shows a top view of a bandage in accordance with the principles of the present invention;

FIG. 2 shows a side cut-away view of another bandage in accordance with the principles of the present invention;

FIG. 3 shows a top view of the bandage of FIG. 2;

FIG. 4 shows yet another bandage in accordance with the principles of the present invention; and

FIG. 5 shows a backing for a bandage in accordance with the principles of the present invention.

### DETAILED DESCRIPTION OF INVENTION

The detailed description set forth below in connection with the appended drawings is intended as a description of various embodiments of the invention and is not intended to represent the only embodiments in which the invention may be practiced. The detailed description includes specific details for the purpose of providing a thorough understanding of the invention. However, it will be apparent to those skilled in the art that the invention may be practiced without these specific details. In some instances, well known structures and components are shown in block diagram form in order to avoid obscuring the concepts of the invention.

In the embodiments described below, it is apparent that one of ordinary skill is able to select appropriate materials from those already known in the art to manufacture the bandages and dressing described herein. Certain materials are known to be more durable than others and, thus, the purpose, size and location of each bandage can be considered when determining what materials to use. Additionally, some bandages require more adhesion properties than normally available and, as is known in the industry, additional spray-on, or after-market adhesives may be used to augment embodiments of the present invention. Similarly, known manufacturing processes and techniques are available for ensuring that any bandages may be produced in a sterile manner and that the pad or gauze can be impregnated with various additives such as antibacterial agents.

As mentioned previously, there have been attempts to address wound management concerns in the area of chronic sores and diabetic ulcers and the like. These techniques have proven to be time consuming, labor intensive and require technical training. Furthermore, as a general rule, the techniques have a large amount of intra-observer variability such that consistent measures across different practitioners are difficult to achieve.

In an acute care situation, such techniques are impractical to implement and alternative measures must be developed. For example, with cardiac catheterization through the femoral artery, it is important to be able to quickly assess the size and variability of any hematoma or other bleeding near the incision site.

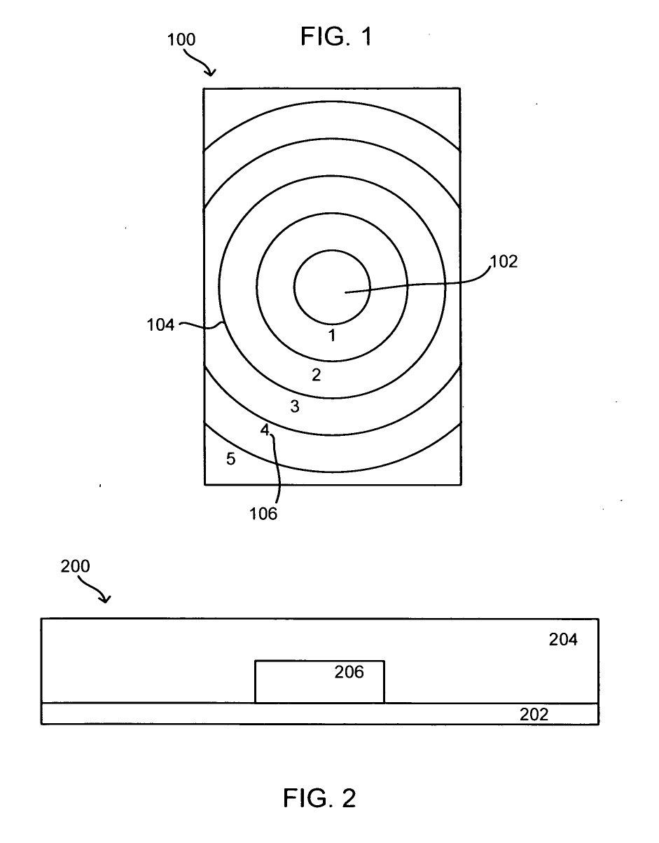

FIG. 1 illustrates a bandage 100 in accordance with the principles of the present invention. A central region 102 includes gauze or other padding that is to be placed over the incision or surgical site. The remaining portion of the bandage 100 is constructed from a transparent material and includes evenly spaced curved markings 104. In the example of the femoral site mentioned briefly above, a bandage of about 10×12 inches may potentially be used. In this application it is useful to be able to measure an underlying hematoma to the closest centimeter. Thus, each of the scale marks 104 has a radius one centimeter greater than its closest inner neighbor. With the pad 102 centered over the incision point, the size of an underlying hematoma can be determined quickly and easily simply by finding the closest line to the outside edge of the hematoma. No estimating of wound area is necessary to properly analyze the condition of the hematoma

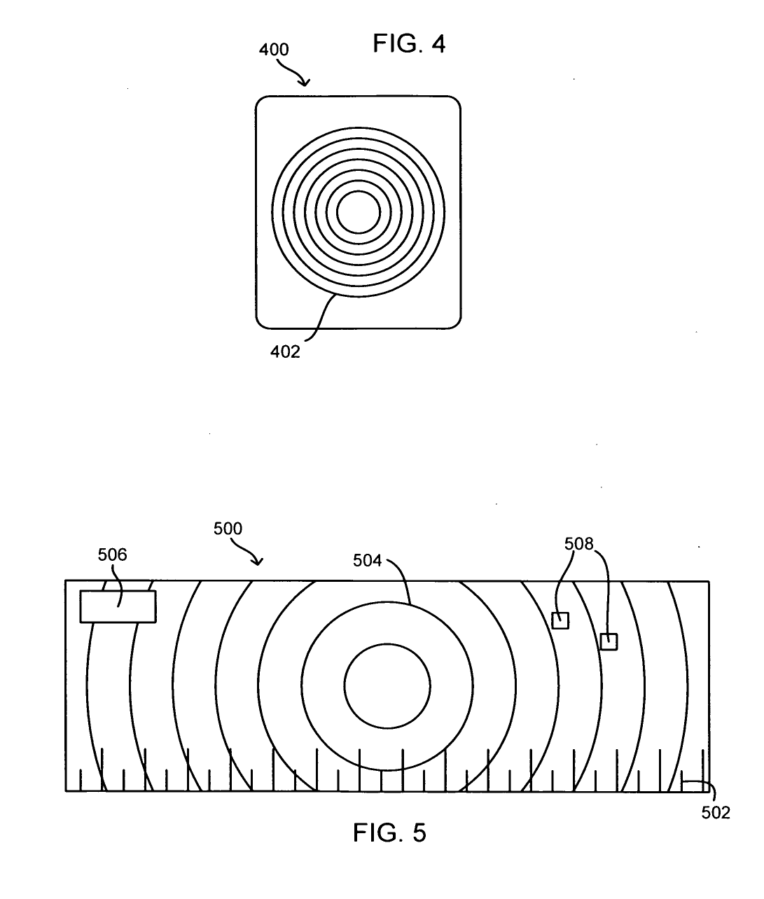

One of ordinary skill will recognize that the size of the bandage 100 can vary dramatically without departing from the scope of the present invention. For example, FIG. 4 shows a smaller bandage 400, having a different shape even, in which the curved scale lines 402 are closer spaced. For example, for an incision near the carotid artery, the size of a hematoma which raises concerns will be different than at the femoral site. Thus, based on the location and the underlying medical principles at work, a bandage may be sized appropriately and also include a scale resolution (i.e., how far apart the curved lines are spaced) that is appropriate as well.

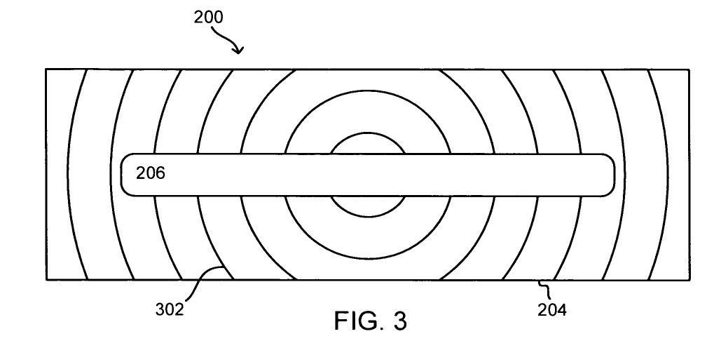

FIGS. 2 and 3 depict another type of bandage 200 in which the pad or gauze 206 is an island shape that runs a substantial length of the bandage 200. This type of bandage may be useful for a longer incision site than a mere point-incision. The principles of the invention, however, remain the same in that curved scale lines 302 are present on a transparent portion of the bandage to allow a practitioner, or the patient, to quickly and easily assess the state of the incision under the bandage using some objective measure. The view of FIG. 2 shows that the bandage 200 includes a backing layer 202 that is removed to reveal a sticky side of a transparent region 204 and the pad area 206.

As described, the bandage is useful when it is in place; however, there are times when the bandage has been removed but it is still important to monitor a condition of the incision site. In particular, once the patient is not under the immediate care of medical personnel, it is still beneficial for that patient to be able to objectively monitor the incision site in a way that is helpful for reporting the condition to their practitioner.

FIG. 5 depicts an exemplary backing portion of a bandage in accordance with the principles of the present invention. The backing 500 is constructed of a transparent material that also includes a series of curved scale lines 504 similar to that of the bandage it backs. The backing 500 may also include a different type scale 502 and a region 506 that can be written on, beneficially in an indelible way. Also included are labels 508 that provide an appropriate identification of the scale line adjacent such labels 508.

In operation, the backing 500 is removed so that the bandage is placed on the patient by a practitioner. As part of the bandage placement process, the practitioner may make appropriate markings in the writable portion 506 indicating the initial size of the hematoma, the patient's name, and emergency number, or a warning size to remind the patient to call if the hematoma ever exceeds a certain size. Additionally, substantially the entire surface of the backing can writable so that the practitioner can place the backing over the bandage and incision site and draw the initial hematoma on the backing. The backing is given to the patient as educational material and to aid in monitoring the incision site.

To use the backing 500, the patient (at some later time that the bandage has been removed) holds the backing over the incision site to monitor the hematoma. With the use of the curved scale lines 504, the patient can make an accurate and objective size determination which can be conveyed to their practitioner.

The labels 508 can be printed on both sides of the backing to ensure visibility from both sides or can even be printed in reverse. In this latter instance, there are many instances in which the incision site results in the use of the backing by the patient requiring the use of a mirror. By printing the labels in reverse, they will appear correctly when viewed in a mirror. Alternatively, by printing the labels in one direction and dark enough to be visible regardless of which way the backing 500 is held, the same backing can be used in a mirror scenario by the patient but also can simply be flipped to be used by a care-giver of the patient. The backing 500 is constructed of a material and of a thickness to provide durable service to a patient for between 3 to 5 days. In at least one embodiment, the backing 500 is flexible enough to temporarily adhere to a patient's incision site through water tension so that by moistening the backing, the patient can have both hands free to observe the incision site.

One of ordinary skill will recognize that the present invention is not limited to a particular sized or shaped bandage nor are the curved scale lines required to have a particular resolution. For example, bandages in accordance with the principles of the present invention may be useful for pacemaker incision sites, abdominal site with a hip-to-hip incision, lumbar punctures, carotid incisions for cardiac catheterization, laparoscopic incisions, IV sites, mole removal, biopsies, cyst removal, basically any incision into subcutaneous tissue in humans and animals. Such a bandage as described herein will allow monitoring of hematomas and ecchymosis as well as other phenomena useful in an acute care situation such as swelling, redness, bruising, drainage and other complications or changes at the incision site. In an embodiment where the curved scale lines are centered around the center of the bandage, the bandage can be cut-to-order in order to customize it to a certain applications.

The curved scale lines on both the backing and the bandage can be constructed to have properties to enhance their visual perceptibility. For example, in some low-light applications, the lines may be reflective, glow in the dark, or respond to ultraviolet radiation (e.g., the black light effect). Thus, various techniques for constructing the lines to increase their contrast with the underlying hematoma are considered within the scope of the present invention.

The previous description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with each claim's language, wherein reference to an element in the singular is not intended to mean “one and only one” unless specifically so stated, but rather “one or more.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

What is claimed is:

1. A bandage comprising:
a transparent portion configured to adhere to an incision site,
a pad centrally located on the transparent portion; and
a plurality of curved scale lines visible on the transparent portion concentrically arranged around the pad and evenly spaced.

2. The bandage of claim 1, wherein each of the lines has a constant radius.

3. The bandage of claim 1, further comprising:
a respective label adjacent each line indicating a radius of that line.

4. The bandage of claim 1, wherein the bandage is substantially rectangular.

5. The bandage of claim 1, wherein the bandage is substantially circular.

6. The bandage of claim 1, wherein the bandage is substantially square.

7. The bandage of claim 1, wherein the pad comprises an island pad extending along a major axis of the bandage.

8. The bandage of claim 1, wherein the pad comprises a circular pad.

9. The bandage of claim 1, further comprising:
a transparent backing, the transparent backing including another plurality of curved scale lines substantially similar to those on the transparent portion.

10. The bandage of claim 9, wherein the transparent backing further includes:
a respective label for each of another plurality of curved scale lines.

11. The bandage of claim 10, wherein each respective label is visually perceptible when viewed from either side of the transparent backing.

12. The bandage of claim 10, wherein each respective label is printed on the backing in reverse.

13. The bandage of claim 9, wherein the transparent backing includes a writable region.

14. A transparent backing for a bandage, comprising:
a plurality of curved scale lines visible on the transparent backing concentrically arranged around a pad of the bandage and evenly spaced.

15. The transparent backing of claim 14, wherein the plurality of curved scale lines are substantially similar to another plurality of curved scale lines on a transparent portion of the bandage.

16. The transparent backing of claim 14, further comprising:
a respective label for each of plurality of curved scale lines.

17. The transparent backing of claim 16, wherein each respective label is visually perceptible when viewed from either side of the transparent backing.

18. The transparent backing of claim 16, wherein each respective label is printed on the backing in reverse.

19. The transparent backing of claim 14, wherein the transparent backing includes a writable region.

20. A bandage comprising:
a transparent portion configured to adhere to an incision site,
a pad centrally located on the transparent portion;
a plurality of curved scale lines visible on the transparent portion concentrically arranged around the pad and evenly spaced; and
a transparent backing, the transparent backing including another plurality of curved scale lines substantially similar to those on the transparent portion.
